(19) United States
(12) Patent Application Publication
(10) Pub. No.: US 2004/0028621 A1
(43) Pub. Date: Feb. 12, 2004

(54) Aerosol device containing a styling composition, and styling process

(75) Inventor: Francois Le Bourhis, Aubervilliers (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/449,083

(22) Filed: Jun. 2, 2003

Related U.S. Application Data
(60) Provisional application No. 60/385,550, filed on Jun. 5, 2002.

(30) Foreign Application Priority Data
May 31, 2002 (FR) 0206733

(51) Int. Cl. A61K 7/00; A61K 9/00

(52) U.S. Cl. 424/47

(57) ABSTRACT

The present invention relates to an aerosol device containing a styling composition in a first compartment and a compressed gas selected from the group consisting of air, nitrogen and carbon dioxide, and mixtures thereof, in a second compartment. The styling composition contains at least one fixing polyurethane in an alcoholic or aqueous-alcoholic cosmetically acceptable medium having at least 5% by weight, relative to the weight of the composition, of at least one C₁-C₄ monohydric alcohol.

### REFERENCE TO RELATED APPLICATIONS

This application claims priority to U.S. provisional application 60/385550 filed Jun. 5, 2002, and to French patent application 0206733 filed May 31, 2002, both incorporated herein by reference in their entirety

### SUMMARY OF THE INVENTION

The present invention relates to an aerosol device comprising a styling composition and a compressed gas, and to a styling process.

### BACKGROUND OF THE INVENTION

Styling compositions, such as lacquers and sprays, packaged in aerosol spray form are generally composed of a liquid phase comprising, in an aqueous, alcoholic or aqueous-alcoholic cosmetically acceptable medium, at least one fixing polymer, and a propellant, which is a liquefied gas under reduced pressure or dissolved in the liquid phase.

Other aerosol devices comprising such a liquid phase exist, but they comprise compressed gases for propelling the liquid phase. However, these compressed-gas devices have the drawback of losing gas pressure over time. Specifically, it is observed that the compressed gases leak out over time or during incorrect use of the container, i.e. when it is held upside-down.

This drawback results in an increasingly poor propulsion of the liquid phase over time and consequently increasingly poor distribution of the liquid phase on the hair.

Another factor that may lead to poor distribution of the liquid phase from these devices is the quality of the spray, which degrades when it is desired to obtain the greatest possible fixing, i.e. when it is desired to increase the amount of polymer that may be dissolved in the said liquid phase.

### DETAILED DESCRIPTION OF THE INVENTION

The inventors have made the surprising discovery that mechanical separation of the liquid phase from the propellant compressed gas, and the use of a particular compressed gas and of a fixing polyurethane, make it possible to solve the problems of leakage and of distribution of the liquid phase on the hair. This particular combination makes it possible to obtain, e.g., fixing and/or shaping with a level of fixing that cannot usually be achieved with this type of product.

One subject of the invention is an aerosol device, preferably a two compartment device, comprising a styling composition which comprises at least one fixing polyurethane, and a compressed gas as described below.

Another subject of the present invention is a styling process.

Other subjects, characteristics, aspects and advantages of the invention will emerge even more clearly on reading the description and the various examples that follow.

The aerosol device according to the invention comprises:

(a) in a first compartment, a styling composition comprising at least one fixing polyurethane in an alcoholic or aqueous-alcoholic cosmetically acceptable medium that comprises at least 5% by weight, relative to the total weight of the said composition, of at least one C₁-C₄ monohydric alcohol, and

(b) in a second compartment, a compressed gas selected from the group consisting of air, nitrogen and carbon dioxide, and mixtures thereof, air being particularly preferred.

The compressed gas is preferably used at a pressure of between 1 and 12 bar and better still between 9 and 11 bar.

For the purposes of the present invention, the term “fixing polyurethane” means polycondensates comprising at least one polyurethane block capable of providing hold to the hairstyle. Useful polycondensates include those that are described in patents EP 0 751 162, EP 0 637 600, FR 2 743 297 and EP 0 648 485, EP 0 656 021, WO 94/03510 and EP 0 619 111, all incorporated herein by reference.

The fixing polyurethanes used in accordance with the invention may be soluble in the cosmetically acceptable medium, especially after neutralization with an organic or mineral base, or alternatively they may form a dispersion in this medium. The dispersion may in this case comprise at least 0.05% surfactant for dispersing the fixing polyurethane and holding it in dispersion.

According to the invention, any type of surfactant may be used in said dispersion, but preferably a nonionic surfactant. The mean particle size of the fixing polyurethane in the dispersion is preferably between 0.1 and 1 micrometres.

By way of example, the fixing polyurethane may be formed by an arrangement of blocks, this arrangement being obtained especially from:

(1) at least one compound which contains two or more than two active hydrogen atoms per molecule;

(2) at least one diol or a mixture of diols containing acid functions or salts thereof; and

(3) at least one di- or polyisocyanate.

Advantageously, the compounds (1) are selected from the group consisting of the group comprising diols, diamines, polyesterols and polyetherols, or mixtures thereof.

The compounds (1) that are preferred are linear polyethylene glycols and linear polypropylene glycols, in particular those obtained by reacting ethylene oxide or propylene oxide with water or diethylene glycol or dipropylene glycol in the presence of sodium hydroxide as catalyst. These polyalkylene glycols generally have a molecular weight of between about 600 and 20 000.

Other preferred organic compounds are those containing mercapto, amino, carboxyl or hydroxyl groups. Among these, mention may be made more particularly of polyhydroxylated compounds such as polyetherdiols, polyesterdiols, polyacetaldiols, polyamidediols, polyesterpolyamidediols, poly(alkylene ether)diols, polythioetherdiols and polycarbonatediols.

The preferred polyetherdiols are, for example, the products of condensation of ethylene oxide, propylene oxide or tetrahydrofuran, the grafted or block products of copolymerization or of condensation thereof, such as mixtures of condensates of ethylene oxide and of propylene oxide, and the products of polymerization of olefins, at high pressure, with alkylene oxide condensates. Suitable polyethers are prepared, for example, by condensation of alkylene oxides and of polyhydric alcohols, such as ethylene glycol, 1,2-propylene glycol and 1,4-butanediol.

The polyesterdiols, polyesteramides and polyamidediols are preferably saturated and are obtained, for example, from the reaction of saturated or unsaturated polycarboxylic acids with polyhydric alcohols, diamines or polyamines. Adipic acid, succinic acid, phthalic acid, terephthalic acid and maleic acid may be used, for example, to prepare these compounds. Polyhydric alcohols that are suitable for preparing polyesters include, for example, ethylene glycol, 1,2-propylene glycol, 1,4-butanediol, neopentyl glycol and hexanediol. Amino alcohols, for example ethanolamine, may also be used. Diamines that are suitable for preparing the polyester amides are ethylenediamine and hexamethylenediamine.

Suitable polyacetals may be prepared, for example, from 1,4-butanediol or hexanediol and formaldehyde. Suitable polythioethers may be prepared, for example, by condensation reaction between thioglycols alone or in combination with other glycols such as ethylene glycol, 1,2-propylene glycol or with other polyhydroxylated compounds. Polyhydroxylated compounds already containing urethane groups, natural polyols, which may be further modified, for example castor oil and carbohydrates, may also be used.

More preferably, the compound of group (1) is a polyesterol, especially a polyesterdiol formed by the reaction of at least one (di)polyol (1ₐ) and at least one acid (1_{b}). The (di)polyol (1ₐ) is chosen in particular from the group comprising neopentyl glycol, 1,4-butanediol, hexanediol, ethylene glycol, diethylene glycol, propylene glycol, butylene glycol, neopentyl glycol and (di)polyethylene glycol. The acid (1_{b}) is chosen in particular from the group comprising phthalic acid, isophthalic acid, adipic acid and (poly)lactic acid.

A hydroxycarboxylic acid such as dimethylolpropanoic acid (DMPA) or a 2,2-hydroxy-methylcarboxylic acid may especially be used as compound (2). In general, compound (2) is useful as a coupling block. Compounds (2) that are preferred include those comprising at least one poly((αhydroxycarboxylicdiol) acid).

The compounds (2) that are particularly preferred in accordance with the invention are those selected from the group consisting of the group comprising 2,2-di(hydroxymethyl)acetic acid, 2,2-dihydroxymethylpropionic acid, 2,2-dihydroxymethylbutyric acid and 2,2-dihydroxymethylpentanoic acid. The di- or polyisocyanate (3) may be chosen in particular from the group comprising hexamethylene diisocyanate, isophorone diisocyanate (IDPI), tolylene diisocyanate, 4,4′-diphenylmethane diisocyanate (DPMD) and 4,4′-dicyclohexylmethane diisocyanate (DCMD), methylenebis(p-phenyl diisocyanate), methylenebis(4-cyclohexyl isocyanate), toluene diisocyanates, 1,5-napthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4- and 2,6-toluene diisocyanates, 2,2′-dichloro-4,4′-diisocyanatodiphenylmethane, 2,4-dibromo-1,5-diisocyanatonaphthalene, 1,4-butane diisocyanate, 1,6-hexane diisocyanate and 1,4-cyclohexane diisocyanate.

The fixing polyurethane may be formed using an additional compound (4) that generally serves to lengthen its chain. These compounds (4) may be selected from the group consisting of the group especially comprising saturated or unsaturated glycols such as ethylene glycol, diethylene glycol, neopentyl glycol or triethylene glycol; amino alcohols such as ethanolamine, propanolamine or butanolamine; heterocyclic, aromatic, cycloaliphatic and aliphatic primary amines; diamines; carboxylic acids such as aliphatic, aromatic and heterocyclic carboxylic acids, for instance oxalic acid, succinic acid, glutaric acid, adipic acid, sebacic acid and terephthalic acid; aminocarboxylic acids. The compounds (4) that are preferred are aliphatic diols.

The fixing polyurethanes used according to the invention may also be formed from additional compounds (5) having a silicone skeleton, such as polysiloxanes, polyalkylsiloxanes or polyarylsiloxanes, especially polyethylsiloxanes, polymethylsiloxanes and polyphenylsiloxanes, optionally comprising hydrocarbon-based chains grafted onto the silicon atoms.

The fixing polyurethanes advantageously used comprise a base repeating unit corresponding to the general formula (I):

—O—B—O—CO—NH—R—NH—CO— (I)

in which:

B is a divalent C₁ to C₃₀ hydrocarbon-based group, this group being optionally substituted with a group comprising one or more carboxylic acid functions and/or one or more sulphonic acid functions, the said carboxylic acid and/or sulphonic acid functions being in free form or partially or totally neutralized with a mineral or organic base, and

R is a divalent group selected from the group consisting of alkylene groups of aromatic type, C₁ to C₂₀ aliphatic groups and C₁ to C₂₀ cycloaliphatic groups, these groups being substituted or unsubstituted.

The group R is advantageously selected from the group consisting of groups corresponding to the following formulae:

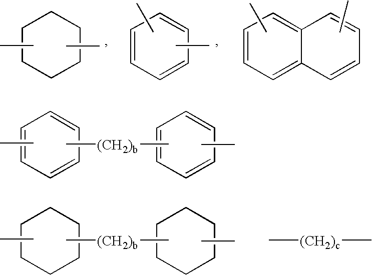

in which b is an integer between 0 and 3, and c is an integer between 1 and 20 and preferably between 2 and 12.

In particular, the group R is preferably selected from the group consisting of hexamethylene, 4,4′-biphenylenemethane, 2,4- and/or 2,6-tolylene, 1,5-naphthylene, p-phenylene and methylene-4,4-bis(cyclohexyl) groups and the divalent group derived from isophorone.

The fixing polyurethane used in the present invention may also advantageously comprise at least one polysiloxane block in its base repeating unit corresponding, for example, to the general formula (II):

—O—P—O—CO—NH—R—NH—CO— (II)

in which:

P is a polysiloxane segment, and

R is a divalent group selected from the group consisting of alkylene groups of aromatic type, C₁ to C₂₀ aliphatic groups and C₁ to C₂₀ cycloaliphatic groups, these groups being substituted or unsubstituted.

Advantageously, the polysiloxane segment P corresponds to the general formula (III) below:

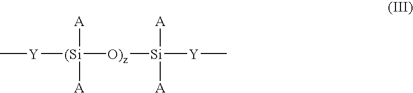

in which:

the groups A, which may be identical or different, are chosen preferably from monovalent C₁ to C₂₀ hydrocarbon-based groups that are free or substantially free of ethylenic unsaturation, and from aromatic groups,

Y represents a divalent hydrocarbon-based group, and

z represents an integer chosen such that the average molecular weight of the polysiloxane segment is between 300 and 10 000.

In general, the divalent group Y is selected from the group consisting of the alkylene groups of formula —(CH₂)ₐ—, in which a represents an integer that may be between 1 and 10.

The groups A may be selected from the group consisting of alkyl groups, in particular methyl, ethyl, propyl, isopropyl, butyl, pentyl, hexyl, octyl, decyl, dodecyl and octadecyl groups; cycloalkyl groups, in particular the cyclohexyl group; aryl groups, especially phenyl and naphthyl; arylalkyl groups, especially benzyl and phenylethyl, and also tolyl and xylyl groups.

Examples of fixing polyurethanes that may especially be mentioned include the dimethylolpropionic acid/isophorone diisocyanate/neopentyl glycol/polyesterdiols polyesterdiols copolymer (also known under the name polyurethane-1, INCI name) sold under the brand name Luviset® PUR by the company BASF, and the dimethylolpropionic acid/isophorone diisocyanate/neopentyl glycol/polyesterdiols/silicone diamine copolymer (also known under the name polyurethane-6, INCI name) sold under the brand name Luviset® Si Pur A by the company BASF.

The said fixing polyurethane is preferably present in an amount of between 0.5% and 20% by weight and better still between 1% and 12% by weight relative to the total weight of the styling composition.

The expression “cosmetically acceptable medium” means a medium that is compatible with the hair, but which also preferably has pleasant odour, appearance and feel.

The alcoholic or aqueous-alcoholic cosmetically acceptable medium may comprise at least one alcohol such as a C₁₋₄ alcohol, for example ethanol or isopropanol, or of a mixture of water and of at least one alcohol, for instance a C₁₋₄ alcohol.

The medium comprises at least 5% by weight, preferably from 5% to 99.9% by weight, better still from 10% to 99% by weight and even more preferably from 20% to 98% by weight, relative to the total weight of the said composition, of a C₁₋₄ monohydric alcohol.

In an aqueous-alcoholic medium, the proportion of water can range between 1% and 95% and preferably between 10% and 90% of the total weight of the composition.

The styling composition may also comprise additives such as silicones in soluble, dispersed or microdispersed form, nonionic, cationic, anionic or amphoteric non-polyurethane fixing polymers, treating active agents, moisturizers, for instance glycerol, UV-screening agents, acids, bases, plasticizers, solubilizers, preserving agents, vitamins and provitamins, colorants, pigments, anionic, cationic, nonionic or amphoteric surfactants, fragrances and anticorrosion agents, and mixtures thereof.

A person skilled in the art knows how to select the optional additives and the amount thereof such that they do not harm the properties of the compositions of the present invention in view of this disclosure.

These additives are preferably present in the composition according to the invention in an amount ranging from 0% to 20% by weight relative to the total weight of the composition.

Preferably, the aerosol device comprises an outer aerosol can comprising an inner pocket hermetically welded to a valve. The composition is introduced into the inner pocket and a compressed gas is introduced between the pocket and the can at a pressure that is sufficient to eject the product in the form of a spray through the orifice of a nozzle. Such a device is sold under the name EP Spray by the company EP-Spray System SA. Preferably the device is a two-compartment device.

The aerosol devices of the invention are preferably used to hold hair lacquers.

The present invention also relates to a styling process, which comprises spraying, applying, atomizing, vaporizing, etc. the styling composition contained in the aerosol device according to the invention onto wet or dry hair.

The examples that follow are given as illustrations of the present invention.

In the examples that follow, all the amounts are indicated as weight percentages relative to the total weight of the composition, except where otherwise mentioned.

### EXAMPLE 1

A styling composition was prepared from the ingredients below:

| | % |
|---|---|
| | by weight |
| Dimethylolpropionic acid/isophorone diisocyanate/neopentyl | 30.00 |
| glycopolyesterdiols/silicone diamine copolymer sold under | |
| the name Luviset ® Si Pur A by the company BASF, | |
| at 30% active material in a 60/10 water/alcohol mixture | |
| Ethyihexyl methoxycinnamate sold under the trade name | 0.05 |
| Parsol ® MCX by the company Roche Vitamins | |
| Fragrance | 0.10 |
| Absolute ethyl alcohol qs | 100 |

The composition prepared above was introduced into an aerosol distribution device sold under the name EP Spray by the company EP Spray System S.A. described above. A valve of reference 6001 format D6 is attached to a standard aerosol can and the diffuser is a vortex-nozzle diffuser.

The pocket is filled with the composition as indicated above. Compressed air is introduced between the pocket and the can.

The composition was vaporized onto dry hair. The spraying takes place in the form of a gentle spray.

After drying, a head of hair with good hold is obtained.

### EXAMPLE 2

A second styling composition was prepared from the ingredients below:

| | % |
|---|---|
| | by weight |
| Dimethylolpropionic acid/isophorone diisocyanate/neopentyl | 30.00 |
| glycopolyesterdiols/silicone diamine copolymer sold under | |
| the name Luviset ® Si Pur A by the company BASF, | |
| at 30% active material in a 60/10 water/alcohol mixture | |
| Ethylhexyl methoxycinnamate sold under the trade name | 0.05 |
| Parsol ® MCX by the company Roche Vitamins | |
| Demineralized water | 18 |
| Fragrance | 0.10 |
| Absolute ethyl alcohol qs | 100 |

The composition prepared above was introduced into an aerosol distribution device sold under the name EP Spray as described in Example 1.

The composition was vaporized onto dry hair.

Results similar to those obtained in the case of Example 1 were obtained.

All references mentioned herein are incorporated herein by reference in their entirety. Where ranges or limits are discussed all values therewithin are specifically included as if written out.

1. An aerosol device comprising:
(a) in a first compartment, a styling composition comprising at least one fixing polyurethane in an alcoholic or aqueous-alcoholic cosmetically acceptable medium that comprises at least 5% by weight, relative to the weight of the said composition, of at least one C₁-C₄ monohydric alcohol, and
(b) in a second compartment, a compressed gas selected from the group consisting of air, nitrogen and carbon dioxide, and mixtures thereof.

2. The device according to claim 1, wherein the compressed gas is air.

3. The device according to claim 1, wherein the pressure of the compressed gas is between 1 and 12 bar.

4. The device according to claim 3, wherein the pressure of the compressed gas is between 9 and 11 bar.

5. The device according to claim 1, wherein the fixing polyurethane comprises a repeating unit corresponding to the general formula (I):
—O—B—O—CO—NH—R—NH—CO— (I)
in which:
B is a divalent C₁ to C₃₀ hydrocarbon-based group, this group being optionally substituted with a group comprising one or more carboxylic acid functions and/or one or more sulphonic acid functions, the said carboxylic acid and/or sulphonic acid functions being in free form or partially or totally neutralized with a mineral or organic base, and
R is a divalent group selected from the group consisting of alkylene groups of aromatic type, C₁ to C₂₀ aliphatic groups and C₁ to C₂₀ cycloaliphatic groups, these groups being substituted or unsubstituted.

6. The device according to claim 1, wherein the fixing polyurethane comprises a repeating unit corresponding to the general formula (II):
—O—P—O—CO—NH—R—NH—CO— (II)
in which:
P is a polysiloxane segment, and
R is a divalent group selected from the group consisting of alkylene groups of aromatic type, C₁ to C₂₀ aliphatic groups and C₁ to C₂₀ cycloaliphatic groups, these groups being substituted or unsubstituted.

7. The device according to claim 5, wherein the fixing polyurethane is a dimethylolpropionic acid/isophorone diisocyanate/neopentyl glycol/polyesterdiols/silicone diamine copolymer.

8. The device according to claim 1, wherein the fixing polyurethane is present in an amount of between 0.5% and 20% by weight relative to the total weight of the composition.

9. The device according to claim 8, wherein the fixing polyurethane is present in an amount of between 1% and 12% by weight relative to the total weight of the composition.

10. The device according to claim 1, wherein the alcoholic or aqueous-alcoholic cosmetically acceptable medium consists of alcohol or of a mixture of at least one alcohol and water.

11. The device according to claim 10, wherein the alcohol is a C₁₋₄ alcohol.

12. The device according to claim 11, wherein the alcohol is ethanol or isopropanol.

13. The device according to claim 1, wherein the styling composition further comprises one or more additives selected from the group consisting of silicones in soluble, dispersed or microdispersed form, nonionic, cationic, anionic or amphoteric non-polyurethane fixing polymers, treating active agents, moisturizers, UV-screening agents, acids, bases, plasticizers, solubilizers, preserving agents, vitamins and provitamins, colorants, pigments, anionic, cationic, nonionic or amphoteric surfactants, fragrances and anticorrosion agents, and mixtures thereof.

14. The device according to claim 1, wherein said styling composition constitutes a hair lacquer.

15. The device according to claim 1, wherein said device is a two-compartment device.

16. A process, wherein the styling composition contained in the aerosol device according to claim 1 is applied onto wet or dry hair from said device.

17. A process according to claim 16, wherein the styling composition contained in the aerosol device according to claim 1 is vaporized onto wet or dry hair from said device.
